(19) United States
(12) Patent Application Publication
(10) Pub. No.: US 2008/0095756 A1
(43) Pub. Date: Apr. 24, 2008

(54) Compositions Comprising Lysostaphin Variants And Methods Of Using The Same

(75) Inventors: Jeffrey Richard Stinson, Brookeville, MD (US); Luba Grinberg, Gaithersburg, MD (US); James Mond, Silver Spring, MD (US)

Correspondence Address: Casimir Jones, S.C., 440 Science Drive, Suite 203, Madison, WI, 53711, US

(73) Assignee: Biosynexus Incorporated, Gaithersburg, MD (US)

(21) Appl. No.: 11/850,150

(22) Filed: Sep. 5, 2007

Related U.S. Application Data
(60) Provisional application No. 60/842,402, filed on Sep. 5, 2006.

(51) Int. Cl. A61K 38/48; A61P 31/04; C12N 9/52

(52) U.S. Cl. 424/94.63; 435/220

(57) ABSTRACT

The present invention relates to compositions comprising lysostaphin variants and methods of using the same. In particular, the present invention provides de-immunized lysostaphin variants and methods of using the same (e.g., to treat microbial infection in or on a subject).

This invention claims priority to U.S. Provisional Patent Application Ser. No. 60/842,402 filed Sep. 5, 2006, hereby incorporated by reference in its entirety.

### FIELD OF THE INVENTION

The present invention relates to compositions comprising lysostaphin variants and methods of using the same. In particular, the present invention provides de-immunized lysostaphin variants and methods of using the same (e.g., to treat microbial infection in or on a subject).

### BACKGROUND OF THE INVENTION

Lysostaphin is a potent antimicrobial agent first identified in Staphylococcus simulans (formerly known as S. staphylolyticus). Lysostaphin is a bacterial endopeptidase capable of cleaving the cross-linking polyglycine bridges in the cell walls of bacteria (e.g., Staphylococci), and is therefore highly lethal thereto. Expressed in a single polypeptide chain, lysostaphin has a molecular weight of approximately 27 kDa.

The cell wall bridges of Staphylococcus aureus, a coagulase positive staphylococcus, contain high levels of glycine (e.g., cross-linked polyglycine bridges), and thus lysostaphin is particularly effective in lysing S. aureus. Lysostaphin is also able to lyse Staphylococcus epidermidis.

S. aureus is a highly virulent human pathogen. It is the cause of a variety of human diseases, ranging from localized skin infections to life-threatening bacteremia and infections of vital organs. If not rapidly controlled, a S. aureus infection can spread quickly from the initial site of infection to other organs. Although the foci of infection may not be obvious, organs particularly susceptible to infection include the heart valves, kidneys, lungs, bones, meninges and the skin (e.g., in burn patients).

Small proteins (e.g., less than about 70 kDa), such as lysostaphin, may have a relatively short half-life in blood after intravenous injection. Lysostaphin's rapid clearance from circulation may reduce its efficacy. At the same time, because it is derived from a bacterial species and therefore foreign to any mammalian species, lysostaphin may also have undesired immunogenicity, which further stimulates its clearance from the blood stream, especially in subjects that have had previous exposure to lysostaphin. Thus, there exists a need for improved means by which the circulating half-life of lysostaphin may be increased without increasing the amount or frequency of administration. For example, it would be desirable to generate variants of lysostaphin that display reduced immunogenicity that retain antimicrobial activity.

### SUMMARY OF THE INVENTION

The present invention relates to compositions comprising lysostaphin variants and methods of using the same. In particular, the present invention provides de-immunized lysostaphin variants and methods of using the same (e.g., to treat microbial infection in or on a subject).

In some embodiments, the present invention provides a composition comprising de-immunized lysostaphin. The present invention provides a number of variant, de-immunized lysostaphin molecules, any one or more of which find use in the compositions and methods of the present invention. In some embodiments, the de-immunized lysostaphin comprises SEQ ID NO. 74. In some embodiments, the de-immunized lysostaphin comprises SEQ ID NO. 108. In some embodiments, the de-immunized lysostaphin comprises a sequence selected from SEQ ID NOs. 73, 75-98, 106, 107 and 109. In some embodiments, a de-immunized lysostaphin of the present invention comprises two or more variant sequences described herein. In some embodiments, the de-immunized lysostaphin is capable of cleaving cross-linked polyglycine bridges in the cell wall peptidoglycan of staphylococci. In some embodiments, the de-immunized lysostaphin is recombinantly produced. In some embodiments, the de-immunized lysostaphin possesses a terminal cysteine. In some embodiments, the de-immunized lysostaphin is conjugated to a water-soluble polymer. In some embodiments, the water-soluble polymer is selected from the group comprising poly(alkylene oxides), polyoxyethylated polyols and poly(vinyl alcohols). In some embodiments, the said poly(alkylene oxide) is PEG. In some embodiments, the de-immunized lysostaphin is a truncated lysostaphin. The present invention is not limited by the type of lysostaphin truncation utilized, so long as the truncated lysostaphin possesses antimicrobial activity. Indeed, a variety lysostaphin truncations can be utilized in the present invention including, but not limited to, lysostaphin truncations described in U.S. Pat. App. Pub. No. 20050118159 and international publication number WO 03/082184, each of which is hereby incorporated by reference in its entirety.

The present invention also provides a pharmaceutical composition for treating microbial infection comprising de-immunized lysostaphin and a pharmaceutically acceptable carrier. In some embodiments, the de-immunized lysostpahin is less immunogenic than non-de-immunized lysostaphin (e.g., as characterized by an immune response (e.g., antibody response (e.g., IgG response)) elicited by the de-immunized lysostaphin compared to non-de-immunized lysostaphin when administered to a subject). In some embodiments, the pharmaceutical composition comprises an antibiotic. The present invention is not limited by the type of antibiotic utilized. Indeed, a variety of antibiotics find use in the compositions and methods of the present invention including, but not limited to, β-lactams, cephalosporins, aminoglycosides, sulfonamides, antifolates, macrolides, quinolones, glycopeptides, polypeptides and combinations thereof.

The present invention also provides a method for the prophylactic or therapeutic treatment of a microbial infection in a subject comprising administering to the subject a pharmaceutical composition comprising de-immunized lysostaphin and a pharmaceutically acceptable carrier, in an amount effective for preventing or treating the infection. In some embodiments, the infection is a bacterial infection. The present invention is not limited by the type of bacterial infection treated or prevented. In some embodiments, the bacterial infection is caused by bacteria from the genus Staphylococcus. In some embodiments, the bacteria comprises Staphylococcus aureus. In some embodiments, the bacteria comprises Staphylococcus epidermidis.

### DESCRIPTION OF THE DRAWINGS

FIG. 1 shows the sequences of SEQ ID NO. 1 through SEQ ID NO. 72 of the present invention.

FIG. 2 shows IgG response in mice 14 days post a single injection with 50 μg of lysostaphin variant combined with 5 μg of cholera toxin (CT) adjuvant.

FIG. 3 shows IgG response in mice injected on day 1 with 2.5 μg of lysostaphin variant combined with 5 μg of CT, boosted on day 15 with a second injection of 2.5 μg of lysostaphin variant combined with 5 μg of CT, and measured 14 days thereafter.

FIG. 4 shows the sequence name and amino acid sequence, EPIMATRIX score, types of mutations present in the variants, effect of substitute interaction and EPIVAX combined rating for various lysostaphin variants generated during development of the present invention.

FIG. 5 shows the activity of various lysostaphin variants.

FIG. 6 depicts expression vector pJSB40.

FIG. 7 shows the amino acid sequence of several T cell epitopes identified herein and modifications made therein, using the nucleic acid sequences shown, to generate de-immunized lysostaphin molecules.

### DEFINITIONS

To facilitate understanding of the invention, a number of terms are defined below.

As used herein, the term “lysostaphin,” refers to amino acid sequence and/or nucleic acid sequence encoding full length lysostaphin or portion thereof, any lysostaphin mutant or variant (e.g., lysostaphin comprising any one of SEQ ID NOs. 1-98), any lysostaphin truncation (e.g., in which one or more amino acids have been removed from the protein's amino terminus, carboxy terminus, or both), and any recombinantly expressed lysostaphin protein, that retains the proteolytic ability, in vitro and in vivo, of proteolytic attack against glycine-containing bridges in the cell wall peptidoglycan of staphylococci. Modified full-length lysostaphin or lysostaphin variants may be generated by post-translational processing of the protein (either by enzymes present in a host cell strain or by means of enzymes or reagents introduced at any stage of the process) or by mutation of the structural gene. Lysostaphin variants, as describe herein, may include deletion, insertion, domain removal, point and replacement/substitution mutations. Lysostaphin includes, for example, lysostaphin purified from S. simulans, Ambicin L (Nutrition 21, Inc.), purified from B. sphaericus, or lysostaphin purified from a recombinant expression system (e.g., described in U.S. Pat. App. No. 20050118159, hereby incorporated by reference in its entirety). Lysostaphin variants (e.g., de-immunized lysostaphin described herein) may also be expressed in a truncated form.

The term “gene” refers to a nucleic acid (e.g., DNA) sequence that comprises coding sequences necessary for the production of a polypeptide, RNA (e.g., including but not limited to, mRNA, tRNA and rRNA) or precursor (e.g., lysostaphin). The polypeptide, RNA, or precursor can be encoded by a full length coding sequence or by any portion of the coding sequence so long as the desired activity or functional properties (e.g., antimicrobial activity) of the full-length or fragment are retained. The term also encompasses the coding region of a structural gene including sequences located adjacent to the coding region on both the 5′ and 3′ ends for a distance of about 1 kb on either end such that the gene corresponds to the length of the full-length mRNA. The sequences that are located 5′ of the coding region and that are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene.

Where “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (e.g., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

The term “promoter,” as used herein, refers to a DNA sequence that facilitates the production of messenger RNA by the process of transcription. A promoter is “operatively linked” to a gene when the initiation of the transcription process at the promoter leads to the production of messenger RNA encoded by that gene.

The term “express,” as used herein, refers to the process by which messenger RNA is transcribed from a DNA template such that the messenger RNA is then translated into the amino acid sequence that forms a protein. Thus, a DNA molecule expresses lysostaphin when it contains nucleotide sequences that may be transcribed into messenger RNA that will be translated into a lysostaphin protein. For the purposes of this invention, the term “express” is essentially equivalent to the term “functionally encode.”

The term “origin of replication,” as used herein, means a DNA sequence that allows an extrachromosomal piece of DNA, such as a plasmid, to duplicate itself independently of chromosomal replication. The origin of replication often binds host cell proteins that participate in DNA replication in the cell.

The term “signal sequence,” as used herein, refers to a DNA sequence that encodes an amino acid sequence that signals the host cell to perform a specific task with the resulting protein. For example, a signal sequence may instruct a host cell to secrete the encoded protein rather than to keep it inside the cell. The term “termination sequence,” as used herein, means a DNA sequence that stops the process of transcription. A termination sequence normally follows the DNA sequence of the gene of interest in a plasmid.

One aspect of the present invention involves transforming a host cell with a recombinant DNA encoding lysostaphin. The term “host cell,” as used herein means any cell, prokaryotic or eukaryotic, including animal and plant cells, that may be transformed or transfected with a recombinant DNA of the invention. In one embodiment of the invention, the host cell is a bacterium, for example, Eschericia coli, Lactococcus lactis, Bacillus sphaericus, and related organisms. Genetic elements in the recombinant DNA of the invention, such as the origin of replication, the promoter, the signal sequence, and the termination sequence are often host cell specific. Thus additional embodiments include recombinant DNA molecules that contain elements for these functions that work in the specific host cell used.

The term “transform,” as used herein, means the introduction of a DNA molecule into a bacterial cell. Bacterial cells are made “competent” when they will readily receive foreign DNA molecules. Methods for making bacterial cells competent and for transforming these competent cells are standard and known to those of skill in the art. Bacteria may also be transformed by electroporation.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acid bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.”

“Substantially homologous” refers to any nucleic acid sequence that can hybridize (e.g., it is the complement of) the single-stranded nucleic acid sequence under conditions of low stringency as described above.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (e.g., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (e.g., gaps) of 20 percent or less as compared to the reference sequence (that does not comprise additions or deletions) for optimal alignment of the two sequences. Optimal alignment of sequences for aligning a comparison window may be conducted by the local homology algorithm of Smith and Waterman (See, e.g., Smith and Waterman, Adv. Appl. Math. 2: 482 (1981)) by the homology alignment algorithm of Needleman and Wunsch (See, e.g., Needleman and Wunsch, J. Mol. Biol. 48:443 (1970)), by the search for similarity method of Pearson and Lipman (See, e.g., Pearson and Lipman, Proc. Natl. Acad. Sci. (U.S.A) 85:2444 (1988)), by computerized implementations of these algorithms (See, e.g., GAP, BESTFIT, FASTA, and TFASTA in the Wisconsin Genetics Software Package Release 7.0, Genetics Computer Group, 575 Science Dr., Madison, Wis.), or by inspection, and the best alignment (e.g., resulting in the highest percentage of homology over the comparison window) generated by the various methods selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “fragment” as used herein refers to a polypeptide that has an amino-terminal and/or carboxy-terminal deletion as compared to the native protein, but where the remaining amino acid sequence relates to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for antimicrobial activity.

As used herein, the terms “restriction endonucleases” and “restriction enzymes” refer to bacterial enzymes, each of which cut double-stranded DNA at or near a specific nucleotide sequence.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets, that specify stop codons (i.e., TAA, TAG, TGA).

As used herein, the term “purified” or “to purify” refers to the removal of contaminants from a sample. For example, lysostaphin can be purified by removal of contaminating non-lysostaphin proteins. The removal of non-lysostaphin molecules results in an increase in the percent of lysostaphin in the sample.

As used herein, the term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule comprising a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

As used herein, the term “administration” refers to the act of giving a drug, prodrug, or other agent, or therapeutic treatment (e.g., composition comprising de-immunized lysostaphin of the present invention) to a subject (e.g., a subject or in vivo, in vitro, or ex vivo cells, tissues, and organs). Exemplary routes of administration to the human body can be through the eyes (ophthalmic), mouth (oral), skin (topical/transdermal), nose (nasal), lungs (inhalant), oral mucosa (buccal), ear, by injection (e.g., intravenously, subcutaneously, intratumorally, intraperitoneally, etc.) and the like.

As used herein, the term “co-administration” refers to the administration of at least two agent(s) (e.g., de-immunized lysostaphin and one or more other agents (e.g., antimicrobial agents)) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., de-immunized lysostaphin) with a carrier, inert or active, making the composition especially suitable for therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives.

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable composition.

For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable compound.

### DETAILED DESCRIPTION OF THE INVENTION

The present invention relates to compositions comprising lysostaphin variants and methods of using the same. In particular, the present invention provides de-immunized lysostaphin variants and methods of using the same (e.g., to treat microbial infection in or on a subject).

Lysostaphin is a potent antibacterial enzyme naturally produced by Staphylococcus simulans. The gene for lysostaphin has been isolated and characterized. Genetic truncations have been made to remove the lysostaphin signal sequence and repetitive elements (the “pre-pro” domain) and for fusing to either an initiating methionine for intracellular expression or a signal sequence (e.g., to permit the secretion of a single species of lysostaphin into the periplasmic space of E. coli (See, e.g., U.S. Patent App. Pub. No. 20050118159, hereby incorporated by reference in its entirety for all purposes)). Lysostaphin has significant value as an anti-staphylococcal therapeutic, however there exist concerns regarding its immunogenicity (e.g., its ability to induce innate and adaptive immune responses (e.g., T-cell mediated immune responses)) and how this may impact its suitability for treatment of human infections. There is little immunogenicity data available about lysostaphin in humans, and those data are largely limited to studies evaluating topical use of lysostaphin. Studies analyzing topical use of lysostaphin have uncovered little evidence for sensitization or antibody formation (See, e.g., Climo et al., 1998. Antimicrob Agents Chemother, vol. 42, p. 1355-60; Schaffner et al., 1967. Yale J Biol Med, vol. 39, p. 215-29; and Schaffner et al., 1967. Yale J Biol Med, vol. 39, p. 230-44).

However, systemic (e.g., intravenous) administration of an agent (e.g., lysostaphin) often produces immunogenic responses in a host not observed when the agent is administered topically. For example, evidence indicated the development of partially-neutralizing antibodies in rabbits administered lysostaphin, although serum antibacterial activity remained (See, e.g., Climo et al., 1998. Antimicrob Agents Chemother, vol. 42, p. 1355-60). Indeed, any potential immunogenicity elicited by a therapeutic protein is of concern because it may result in reduced efficacy (e.g., due to more rapid clearance or production of neutralizing antibodies) and potentially dangerous allergic responses (e.g., anaphylaxis) as reported for streptokinase and asparaginase (See, e.g., Schellekens, 2003, Nephrol Dial Transplant, vol. 18, p. 1257-1259).

Accordingly, experiments were conducted during development of the present invention in order to generate lysostpahin variants that retained antimicrobial activity that also were “de-immunized.” As used herein, the term “de-immunized” when used in reference to lysostaphin, relates to lysostaphin (e.g., lysostaphin variants, derivatives and/or homologues thereof) wherein the specific removal and/or modification of T-cell epitopes and/or domains has occurred. The term “de-immunized” is well known in the art and, among other things, has been employed for the removal of T-cell epitopes from other therapeutic molecules (e.g., antibodies; See, e.g., WO 98/52976 or WO 00/34317, each of which is hereby incorporated by reference in its entirety).

Humoral antibody formation requires the cooperation of helper T-cells with antigen specific B-cells. To reduce immunogenicity of a molecule, one approach is to reduce the ability of the antigen to interact with and stimulate B-cells and/or reduce their ability to stimulate helper T-cells. The identification of B-cell epitopes is problematic, however, given the fact that they are of indeterminate length, and often dependent on the tertiary structure of the target antigen. T cell epitopes, in contrast, are short (9-15 amino acid), linear peptides (See, e.g., Mol Immunol. 2006 43(13):2037-44). In addition, evidence suggests that reduction of T-cell activation is easier to achieve and has the ability to greatly impact antibody production (See, e.g., Tangri et al., 2005 J. Immunology, vol 174, p 3187-3196). The amino acid sequences that comprise the antigenic determinants that stimulate T-cells are referred to as T-cell epitopes and are displayed in the context of major histocompatibility complex (MHC) molecules on antigen presenting cells. Altering the ability of T cell eptiopes to bind MHC molecules (e.g., inhibiting the binding of the epitope to the MHC molecule, or, altering the affinity between the epitope and the MHC molecule, or, altering the epitope in a manner such that the epitope's orientation is altered while within the binding region of the MHC molecule, or altering the epitope in such a way that its presentation by the MHC molecule is altered) has the potential to render the altered epitopes unable to or less able to stimulate an immunogenic response (e.g., stimulate helper T-cells and B cell responses). Accordingly, using the methods described herein, T-cell epitopes of lysostaphin were identified and subsequently altered in an effort to reduce the immunogenicity of lysostaphin and its ability to induce humoral antibody responses (See, e.g., Examples 1-3).

Thus, de-immunization involves, in accordance with the invention, the identification, modification and/or removal of T-cell epitopes, preferably helper T-cell epitopes. In this context, the term T-cell epitope relates to T-cell epitopes comprising small peptides that are recognized by T-cells in the context of MHC class I and/or class II molecules.

Methods for the identification of T-cell epitopes are known in the art (See, e.g., WO 98/52976, WO 00/34317, and U.S. Pat. App. Pub. No. 20040180386, each of which is hereby incorporated by reference in its entirety) and are, inter alia, described herein. Various methods of identification include, but are not limited to, peptide threading, peptide-MHC binding, human T-cell assays, analysis of cytokine expression patterns, ELISPOT assays, class II tetramer epitope mapping, search of MHC-binding motif databases and the additional removal/modification of T-cell epitopes.

Having identified T cell epitopes by application of the above-recited technologies, these can be eliminated, substituted and/or modified from lysostaphin or fragment(s) thereof (e.g., a sequence of about 7 amino acids to one amino acid short of the full length lysostaphin molecule), usually by one or more amino acid substitutions within an identified MHC binding peptide; as further described herein. In some embodiments, one or more amino acid substitutions are generated that eliminate or greatly reduce binding to MHC class I and/or class II molecules, or alternatively, altering the MHC binding peptide to a sequence that retains its ability to bind MHC class I or class II molecules but fails to trigger T cell activation and/or proliferation.

Mature lysostaphin has been shown to have two functional domains, a C-terminal domain of 92 residues that binds the S. aureus outer cell wall and the N-terminal active site comprising endopeptidase activity (See, e.g., Baba and Schneewind. 1996, Embo J, vol. 15, p. 4789-4797). Lysostaphin has not been successfully crystallized in part due to the differing solvent characteristics of its two separate domains. Thus, prior to the development of the present invention, there existed little detailed information about structure/function relationships for this protein.

During development of the present invention, homology based models were generated using Swiss-Prot modeling software (See, e.g., Guex and Peitsch, 1997. Electrophoresis 18: 2714-2723; Peitsch, 1995 Bio/Technology 13: 658-660; and Schwede et al., 2003. Nucleic Acids Research 31: 3381-3385) and crystal structure coordinates of two proteins (deposited in the Protein Data Base (PDB) at the National Center for Biotechnology Information (NCBI)) with homology to the two domains of lysostaphin. The analysis of two separate proteins each displaying homology to one of the two domains of lysostaphin was conducted in order to provide insight into the structure, folding, and potential immunogenic properties (e.g., T cell epitopes) of lysostaphin.

The N-terminal enzymatic domain of lysostaphin was modeled on LytM (PDB accession code:1QWY) (See, e.g., J Mol. Biol. 2004 Jan. 16; 335(3):775-85;), a zinc binding endopeptidase that has greater than 65% homology at the amino acid level to the enzymatic domain of lysostaphin. The C-terminal targeting domain of lysostaphin was modeled on ALE1 (PDB accession code: 1R77) (See, e.g., J Biol. Chem. 2006; 281(1):549-58), a cell wall endopeptidase (peptidylglycan hydrolase) made by Staphylococcus capitis that has >85% homology to the cell wall binding domain of lysostaphin. Accordingly, the present invention provides “de-immunized” lysostaphin variants (e.g., using site directed mutagenesis) generated in part on the structural information obtained from homology-based modeling as well as immunogenic epitope predictions generated via analysis using a modeling algorithm (e.g., EPIMATRIX algorithm, EPIVAX, Inc., Providence, R.I.).

During development of the present invention, an analysis was undertaken to characterize overlapping 12 amino acid peptide sequences across the entire lysostaphin sequence. EPIVAX (EPIVAX, Inc., Providence, R.I.) developed the EPIMATRIX algorithm and has successfully used this system to identify T-cell epitopes in a variety of proteins. The 12-mer peptides were analyzed against 8 common human MHC class II alleles for their ability to be bound by any of the class II molecules. Peptide sequences with resulting EPIMATRIX Z-Scores ≧1.64 were selected for further evaluation. Forty-nine such frames were identified as having at least one “hit,” many of which fell in close proximity to each other to form a “cluster.” Eight such clusters were identified that contained 79% of the total number of predicted hits. Of these, four clusters (LYS030, LYS070, LYS108, and LYS219) contained the highest number of positive “hits.”

To evaluate the immunogenicity of each of these 8 predicted clusters, an ELISpot assay using lysostaphin-exposed blood was performed. Briefly, a microtiter plate was coated with anti-lysostaphin antibody and then the various lysostaphin peptides were added. Human peripheral blood mononuclear cells (PBMC) were added to the wells, and interferon-γ production was quantified. The level of IFN-γ production indicated the level of T-cell activation elicited by the various peptides and correlated to their levels of immunogenicity. The results of these assays revealed that the regions with the highest predicted immunogenic potential (LYS030, LYS070, LYS108, and LYS219) contained significant T-cell epitopes.

Accordingly, a population of lysostaphin variants were generated in which peptide sequences within these clusters were mutated so as to reduce immunogenicity while concurrently leaving unaltered the antimicrobial activity of the lysostaphin variant. Although an understanding of the mechanism is not necessary to practice the present invention and the present invention is not limited to any particular mechanism of action, in some embodiments, mutations (e.g., amino acid substitutions) in positions identified as likely to contribute to MHC class II molecule binding results in a lysostaphin molecule with substantially reduced immunogenictiy compared to a wild-type lysostaphin molecule. In addition, variant peptides were identified comprising structurally conservative changes that decreased epitope immunogenicity (e.g., ability to bind MHC molecules) of lysostaphin that comprised a smaller chance of negatively affecting bacteriocidal activity. For example, in some embodiments, the present invention provides lysostaphin variants characterized by 3D modeling studies performed to determine whether or not the amino acid changes would have detrimental effects on the overall structure of the variants.

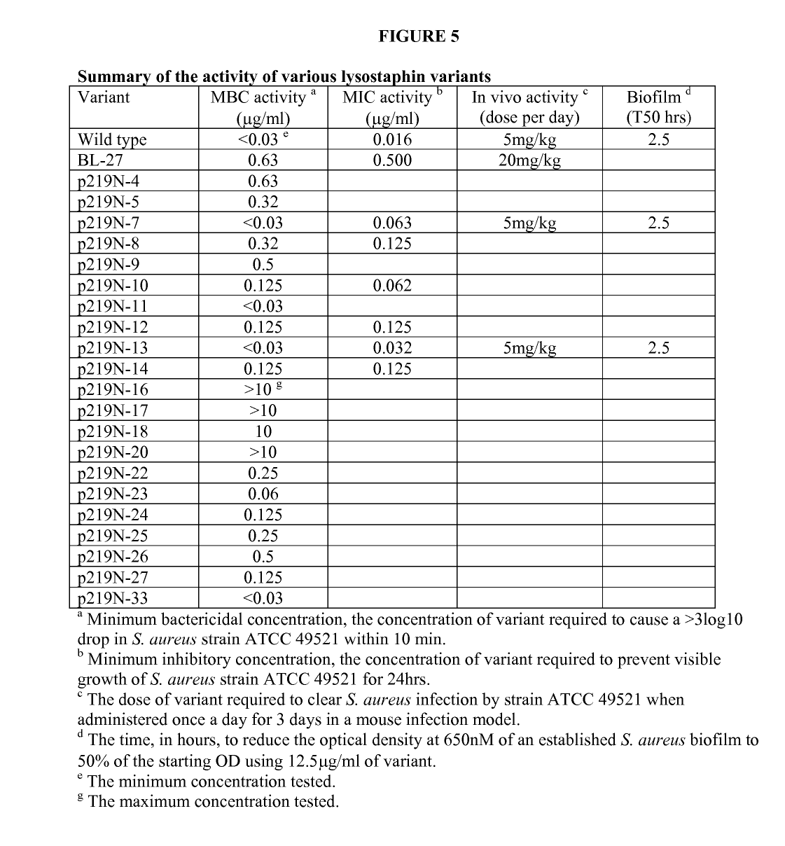

The present invention provides a variety of lysostaphin variants, comprising modification (e.g., mutation (e.g., amino acid substitution)) of immunogenic “hotspots,” that retain antimicrobial (e.g., bactericidal) activity while concurrently displaying reduced immunogenicity (e.g., as measured by anti-lysostaphin antibody production (See, e.g., Examples 1-5, and FIG. 5)).

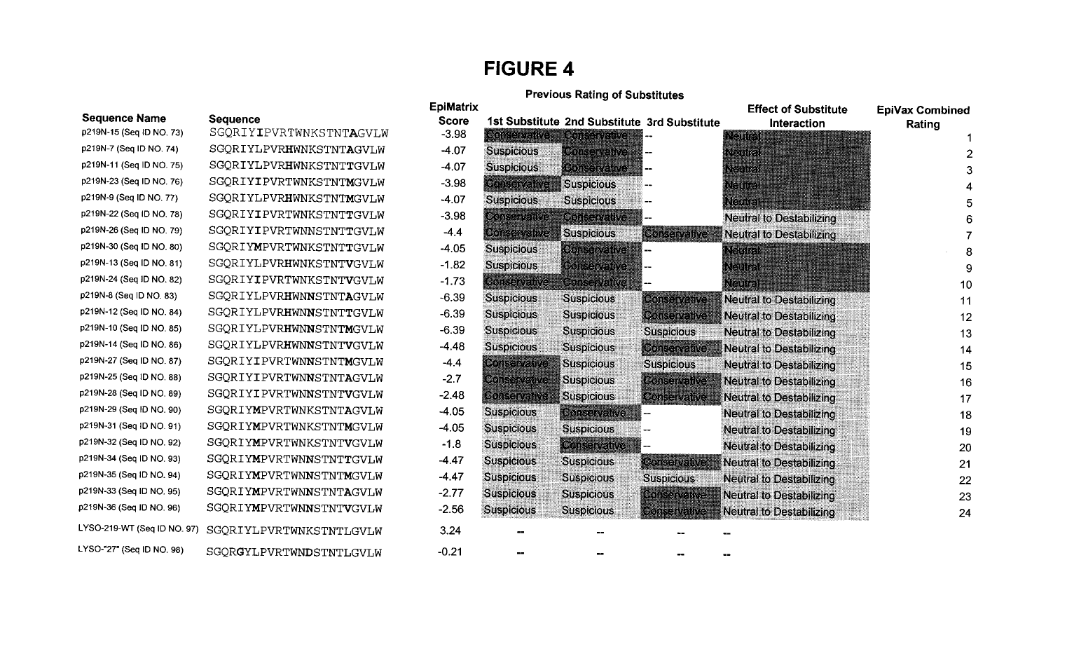

In some embodiments, the present invention provides plasmids (e.g., prokaryotic expression plasmids) comprising nucleic acid sequence encoding variant (e.g., de-immunized) lysostaphin molecules (e.g. that retain antimicrobial activity). The present invention is not limited to any particular lysostaphin variant. Indeed, a variety of variants are provide by the present invention including, but not limited to, those described in Examples 2-4, and FIGS. 4 and 5. In some embodiments, a lysostaphin variant comprises a single amino acid substitution (e.g., any one of the amino acid substitutions described herein) when compared with wild-type sequences (e.g., a mutation within an identified cluster comprising T cell eptiopes). In some embodiments, a lysostaphin variant comprises two amino acid substitutions. In some embodiments, a lysostaphin variant comprises three amino acid substitutions. In some embodiments, a lysostaphin variant comprises four or more amino acid substitutions. In some embodiments, a lysostaphin variant comprises a combination of amino acid substitutions described herein (e.g., in FIG. 4). In some preferred embodiments, a lysostaphin variant comprises one or more amino acid substitutions in the C-terminal targeting/binding domain. In some embodiments, a lysostaphin variant comprises one or more amino acid substitutions in the N-terminal enzymatic domain.

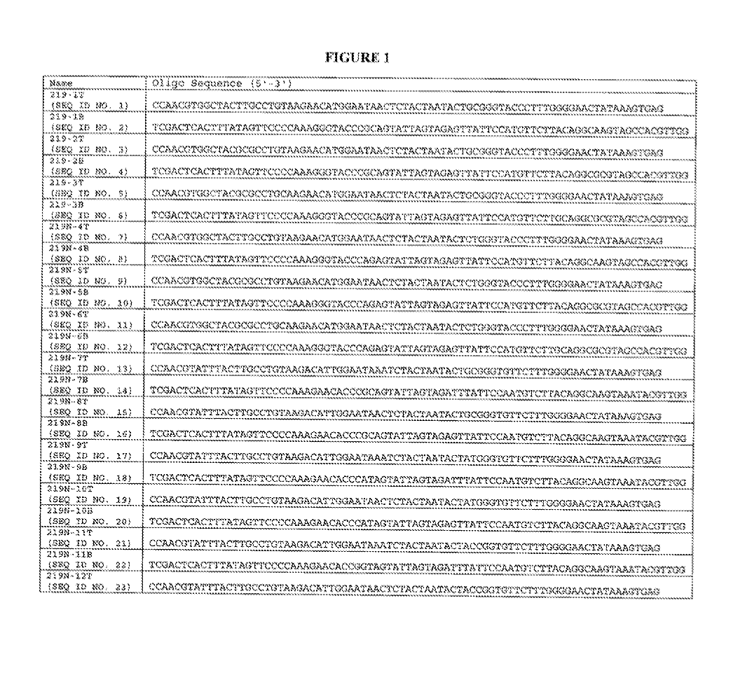

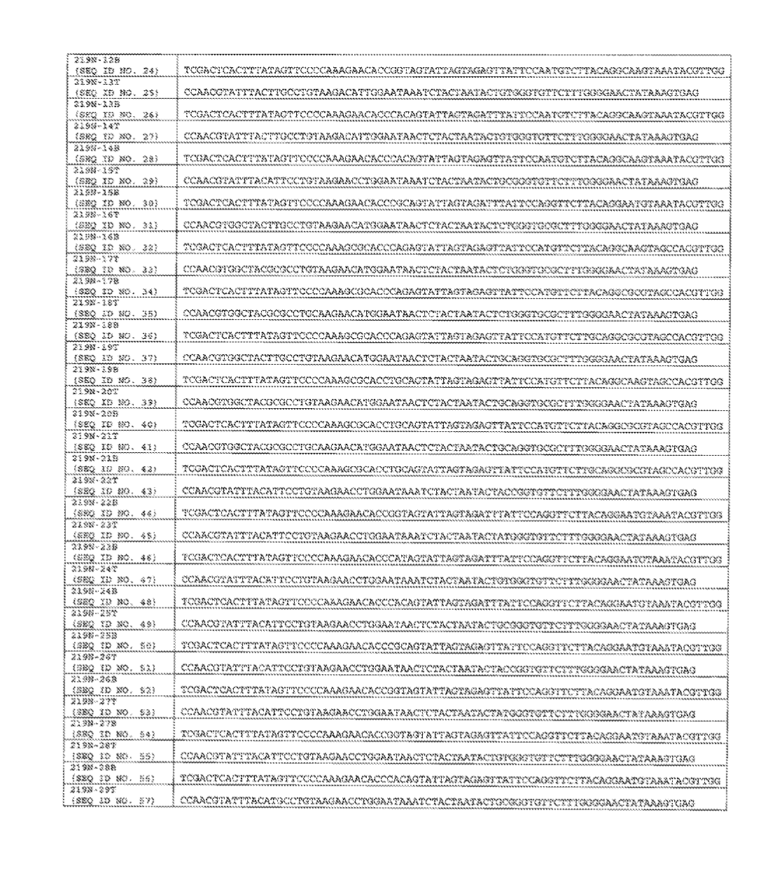

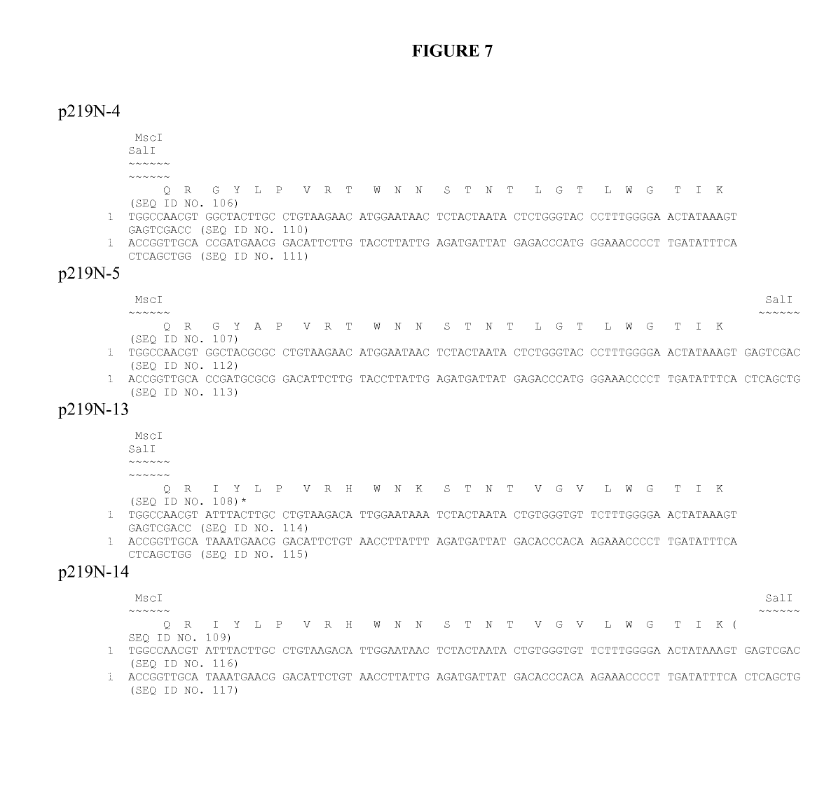

In some embodiments, the present invention provides expression vectors (e.g., plasmids) comprising nucleic acid sequence encoding lysostaphin variants (e.g., that display both reduced immunogenicity (as measured by anti-lysostaphin antibody production) and bactericidal activity). In some embodiments, the lysostaphin variant comprises other domains (e.g., encoded by nucleic acid sequence within the expression vector) that provide for purification means (e.g., histidine stretches). In some embodiments, an expression vector of the present invention comprises nucleic acid sequence described in FIG. 1 (e.g., comprises nucleic acid sequence of one or more of SEQ ID NOs. 1-72 or 110-117) that encode lysostaphin variants (e.g., de-immunized lysostaphin variants (e.g., comprising amino acid sequence described in FIGS. 4 and 7)).

It is immediately evident to a person skilled in the art that regulatory sequences may be added to a nucleic acid molecule encoding a lysostaphin variant of the present invention. For example, promoters, transcriptional enhancers and/or sequences that allow for induced expression of lysostaphin variants may be employed. For example, one suitable inducible system is a tetracycline-regulated gene expression system (See, e.g., Gossen and Bujard, Proc. Natl. Acad. Sci. USA 89 (1992), 5547-5551; and Gossen et al., Trends Biotech. 12 (1994), 58-62). In some embodiments, the inducible system comprises an isopropyl b-D-thiogalactoside (IPTG) inducible promoter.

In some embodiments, a lysostaphin variant comprises one or more of the amino acid mutations depicted in FIG. 4 (SEQ ID NOs. 73-98) and/or FIG. 7 (SEQ ID NOs. 106-109).

The present invention is not limited to any particular type of mutation. Indeed, a variety of mutations may be made (e.g., to generate a lysostaphin variant with reduced immunogenicity) including, but not limited to, amino acid exchange(s), insertion(s), deletion(s), addition(s), substitution(s), inversion(s) and/or duplication(s). These mutations/modification(s) also comprise conservative and/or homologue amino acid exchange(s). Guidance concerning how to make phenotypically/functionally silent amino acid substitution has been described (See, e.g., Bowie (1990), Science 247, 1306-1310).

The present invention also relates to lysostaphin variants that comprise amino acid sequence that is at least 60%, more preferably at least 70%, more preferably at least 80%, more preferably 90%, more preferably at least 95% and most preferably 99% identical or homologous to the polypeptide sequences shown in FIG. 4 (SEQ ID NOs. 73-98).

In some embodiments, a lysostaphin variant of the present invention elicits less than 90%, more preferably less than 80%, more preferably less than 70%, more preferably less than 60%, more preferably less than 50%, more preferably less than 40%, more preferably less than 30%, more preferably less than 20%, and even more preferably less than 10% of the immune response (e.g., as measured by anti-lysostaphin antibody titers) elicited by non-de-immunized lysostaphin.

In some embodiments, the present invention provide a pharmaceutical composition comprising a lysostaphin variant of the present invention. For example, in some embodiments, the present invention provides a composition comprising a lysostaphin variant and a pharmaceutically acceptable carrier.

In some embodiments, the present invention provides a lysostaphin variant (e.g., de-immunized lysostaphin) that may be used in a pharmaceutical composition for treatment or prevention of staphylococcal infection (e.g., of the skin, of a wound, or of an organ) or as a therapy for various active S. aureus infections. In preferred embodiments, a pharmaceutical composition of the present invention comprises a therapeutically effective amount of a lysostaphin of the invention, together with a pharmaceutically acceptable carrier. The present invention is not limited by the types of pharmaceutically acceptable carrier utilized. Indeed, a variety of carriers are well known in the art including, but not limited to, sterile liquids, such as water, oils, including petroleum oil, animal oil, vegetable oil, peanut oil, soybean oil, mineral oil, sesame oil, and the like. Saline solutions, aqueous dextrose, and glycerol solutions can also be employed as liquid carriers, particularly for solution preparations for injection. Suitable pharmaceutical carriers are described in Remington's Pharmaceutical Sciences, 18th Edition (13), which is herein incorporated by reference in its entirety.

A therapeutically effective amount is an amount reasonably believed to provide some measure of relief, assistance, prophylaxis, or preventative effect in the treatment of infection. A therapeutically effective amount may be an amount believed to be sufficient to block a bacterial colonization or infection. Similarly, a therapeutically effective amount may be an amount believed to be sufficient to alleviate (e.g., eradicate) an existing bacterial infection.

A pharmaceutical composition of the present invention may be particularly useful in preventing, ameliorating and/or treating bacterial infection.

The compositions of the invention may be administered locally (e.g., topically) or systemically (e.g., intravenously). Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

In accordance with this invention, the terms “treatment”, “treating” and the like are used herein to generally mean obtaining a desired pharmacological and/or physiological effect. The effect may be prophylactic in terms of completely or partially preventing an infection and/or may be therapeutic in terms of completely or partially treating (e.g., eradicating) a bacterial infection. The term “treatment” as used herein includes: (a) preventing bacterial infection from occurring in a subject (e.g., that may be predisposed to infection (e.g., nosocomial infection) but has not yet been diagnosed as having infection); (b) inhibiting bacterial infection; and/or (c) relieving infection (e.g., completely or partially reducing the presence of bacteria responsible for infection.

Staphylococcal infections, such as those caused by S. aureus, are a significant cause of morbidity and mortality, particularly in settings such as hospitals, schools, and infirmaries. Patients particularly at risk include infants, the elderly, the immunocompromised, the immunosuppressed, and those with chronic conditions requiring frequent hospital stays. Patients also at risk of acquiring staphylococcal infections include those undergoing inpatient or outpatient surgery, those within an Intensive Case Unit (ICU), on continuous hemodialysis, with HIV infection, with AIDS, burn victims, people with diminished immunity (e.g., resulting from drug treatment or disease), the chronically ill or debilitated patients, geriatric subjects, infants with immature immune systems, and people with intravascular (e.g., implanted) devices. Thus, in some embodiments, a composition comprising a lysostaphin variant is administered to any one of these types of subject as well as to other subjects that have or are susceptible to bacterial infection (e.g., caused by S. aureus or S. epidermidis).

In some embodiments, a lysostaphin variant of the present invention is formulated as either an aqueous solution, semi-solid formulation, or dry preparation (e.g., lyophilized, crystalline or amorphous, with or without additional solutes for osmotic balance) for reconstitution. Formulations may be in, or reconstituted in, for example, a non-toxic, stable, pharmaceutically acceptable, aqueous carrier medium, at a pH of about 3 to 8, typically 5 to 8, for administration by conventional protocols and regimes or in a semi-solid formulation such as a cream. Delivery can be via, for example, ophthalmic administration, intravenous (iv), intramuscular, subcutaneous or intraperitoneal routes or intrathecally or by inhalation or used to coat medical devices, catheters and implantable devices, or by direct installation into an infected site so as to permit blood and tissue levels in excess of the minimum inhibitory concentration (MIC) of the active agent to be attained (e.g., to effect a reduction in microbial titers in order to cure, alleviate or prevent an infection). In some embodiments, the antimicrobial agent is formulated as a semi-solid formulation, such as a cream (e.g., that is used in a topical or intranasal formulation).

Furthermore, the lysostaphin variant can be co-administered, simultaneously or alternating, with other antimicrobial agents so as to more effectively treat an infectious disease. Formulations may be in, or be reconstituted in, semi-solid formulations for topical, ophthalmic, or intranasal application, liquids suitable for ophthalmic administration, bolus iv or peripheral injection or by addition to a larger volume iv drip solution, or may be in, or reconstituted in, a larger volume to be administered by slow iv infusion. For example, a lysostaphin variant can be administered in conjunction with antibiotics that interfere with or inhibit cell wall synthesis, such as penicillins, nafcillin, and other alpha- or beta-lactam antibiotics, cephalosporins such as cephalothin, aminoglycosides, sulfonamides, antifolates, macrolides, quinolones, glycopepetides such as vancomycin and polypeptides. In some embodiments, a lysostaphin variant is administered in conjunction with one or more antibiotics that inhibit protein synthesis (e.g., aminoglycosides such as streptomycin, tetracyclines, and streptogramins). The present invention is not limited by the type of agent co-administered with de-immunized lysostaphin. Indeed, a variety of agents may be co-administered including, but not limited to, those agents described in U.S. Pat. Nos. 6,028,051, 6,569,830, and 7,078,377 and U.S. patent application Ser. Nos. 10/414,566, 11/445,289, and 11/494,887, each of which is hereby incorporated by reference in its entirety. In some embodiments, a lysostaphin variant is administered with monoclonal antibodies; other non-conjugated antibacterial enzymes such as lysostaphin, lysozyme, mutanolysin, and cellozyl muramidase; peptides (e.g., defensins); and lantibiotics (e.g., nisin); or any other lanthione-containing molecules (e.g., subtilin).

Agents co-administered with a lysostaphin variant may be formulated together with the lysostaphin variant as a fixed combination or may be used extemporaneously in whatever formulations are available and practical and by whatever routes of administration are known to provide adequate levels of these agents at the sites of infection.

In preferred embodiments, lysostaphin variants according to the present invention possess at least a portion of the antimicrobial activity of the corresponding non-de-immunized antimicrobial agent. A lysostaphin variant of the present invention may be administered in increased dosages and/or at less frequent intervals due to the decreased immunogenicity. In some embodiments, a lysostaphin variant retains at least 10% of the activity of the non-de-immunized antimicrobial agent. In some embodiments, a lysostaphin variant retains at least 20% of the activity of the non-de-immunized antimicrobial agent. In some embodiments, a lysostaphin variant retains at least 30% of the activity of the non-de-immunized antimicrobial agent. In some embodiments, a lysostaphin variant retains at least 40% of the activity of the non-de-immunized antimicrobial agent. In some embodiments, a lysostaphin variant retains at least 50% of the activity of the non-de-immunized antimicrobial agent. In some embodiments, a lysostaphin variant retains at least 60% of the activity of the non-de-immunized antimicrobial agent. In some embodiments, a lysostaphin variant retains at least 70% of the activity of the non-de-immunized antimicrobial agent. In some embodiments, a lysostaphin variant retains at least 80% of the activity of the non-de-immunized antimicrobial agent. In some embodiments, a lysostaphin variant retains at least 90% of the activity of the non-de-immunized antimicrobial agent. In some embodiments, a lysostaphin variant retains 90% or more (e.g., 95%, 97%, 99% or more) of the activity of the non-de-immunized antimicrobial agent.

Suitable dosages and regimes of a de-immunized lysostaphin may vary with the severity of the infection and the sensitivity of the infecting organism and, in the case of combination therapy, may depend on the particular agent (e.g., anti-staphylococcal agent) co-administered. Dosages may range from about 0.05 to about 500 mg/kg/day (e.g., in some embodiments, range from 0.1-10 mg/kg/day, in some embodiments, range from 10-100 mg/kg/day, in some embodiments, range from 100-200 mg/kg/day, in some embodiments, range from 200-400 mg/kg/day, in some embodiments, range from 400-500 mg/kg/day), although higher (e.g., 500-1000 mg/kg/day) or lower (e.g., 0.1-0.5 mg/kg/day doses may be provided, given as single or divided doses, or given by continuous infusion. In some embodiments, de-immunized lysostaphin is administered once a day, twice a day, three times a day or more frequently (e.g., four or more times a day). In some embodiments, de-immunized lysostaphin is administered once a week, twice a week, or every other day. In some embodiments, de-immunized lysostaphin is administered once every other week, once a month, once every two months, once every three months, once every four months, once every five months, once every six months, once every 9 months, once every year or less frequently.

In some embodiments, a de-immunized lysostaphin of the present invention may be further modified in order to further decrease immunogenicity of the lysostaphin molecule while retaining antimicrobial activity. For example, in some embodiments, a de-immunized lysostaphin is conjugated to a water soluble polymer. The present invention is not limited by the type of water soluble polymer to which a de-immunized lysostaphin is conjugated. Indeed, a variety of water soluble polymers may be utilized including, but not limited to, poly(alkylene oxides), polyoxyethylated polyols and poly(vinyl alcohols). Poly(alkylene oxides) include, but are not limited to, polyethylene glycols (PEGs), poloxamers and poloxamines. The present invention is not limited by the type of conjugation utilized (e.g., to connect a de-immunized lysostaphin to one or more water-soluble polymers (e.g. PEG)). In some embodiments, a poly(alkylene oxide) is conjugated to a free amino group via an amide linkage (e.g., formed from an active ester (e.g., the N-hydroxysuccinimide ester)) of the poly(alkylene oxide). In some embodiments, an ester linkage remains in the conjugate after conjugation. In some embodiments, linkage occurs through a lysine residue present in the de-immunized lysostaphin molecule. In some embodiments, conjugation occurs through a short-acting, degradable linkage. The present invention is not limited by the type of degradable linkage utilized. Indeed, a variety of linkages are contemplated to be useful in the present invention including, but not limited to, physiologically cleavable linkages including ester, carbonate ester, carbamate, sulfate, phosphate, acyloxyalkyl ether, acetal, and ketal linkages. In some embodiments, de-immunized lysostaphin is conjugated to PEG utilizing any of the methods, reagents and/or linkages described in U.S. Pat. Nos. 4,424,311; 5,672,662; 6,515,100; 6,664,331; 6,737,505; 6,894,025; 6,864,350; 6,864,327; 6,610,281; 6,541,543; 6,515,100; 6,448,369; 6,437,025; 6,432,397; 6,362,276; 6,362,254; 6,348,558; 6,214,966; 5,990,237; 5,932,462; 5,900,461; 5,739,208; 5,446,090 and 6,828,401; and WO 02/02630 and WO 03/031581, and U.S. Pat. App. No. 60/786,188, each of which is herein incorporated by reference in its entirety. In some embodiments, a de-immunized lysostaphin-water soluble polymer conjugate of the present invention is produced by a third party (e.g., NEKTAR, San Carlos, Calif.). In some embodiments, the conjugate comprises a cleavable linkage present in the linkage between the polymer and de-immunized lysostaphin (e.g., such that when cleaved, no portion of the polymer or linkage remains on the de-immunized lysostaphin molecule). In some embodiments, the conjugate comprises a cleavable linkage present in the polymer itself (e.g., such that when cleaved, a small portion of the polymer or linkage remains on the de-immunized lysostaphin molecule).

In some embodiments, a de-immunized lysostaphin of the present invention is utilized for the treatment and/or prevention of a biofilm (e.g., as described in U.S. Pat. App. Pub. No. 20030215433 and international application WO 03/082148, each of which is hereby incorporated by reference in its entirety). In some embodiments, a de-immunized lysostaphin of the present invention is utilized for nasal applications (e.g., as described in U.S. Pat. App. Pub. No. 20030211995, hereby incorporated by reference in its entirety). In some embodiments, a de-immunized lysostaphin of the present invention is utilized for topical applications (e.g., as described in U.S. Pat. App. Pub. No. 20040192581, hereby incorporated by reference in its entirety). In some embodiments, a de-immunized lysostaphin of the present invention is produced utilizing a high yield production method (e.g., comprising sub-step processes of fermentation, clarification, hydrophobic charge induction chromatography, and purification (e.g., using chromatography and/or dialysis)), as described, for example, in U.S. Pat. App. No. 60/790,698, hereby incorporated by reference in its entirety.

The present invention is further illustrated by the following examples that teach those of ordinary skill in the art how to practice the invention. The following examples are merely illustrative of the invention and disclose various beneficial properties of certain embodiments of the invention. The following examples should not be construed as limiting the invention as claimed.

### EXPERIMENTAL

The following examples are provided in order to demonstrate and further illustrate certain preferred embodiments and aspects of the present invention and are not to be construed as limiting the scope thereof.

### Example 1

### Materials and Methods

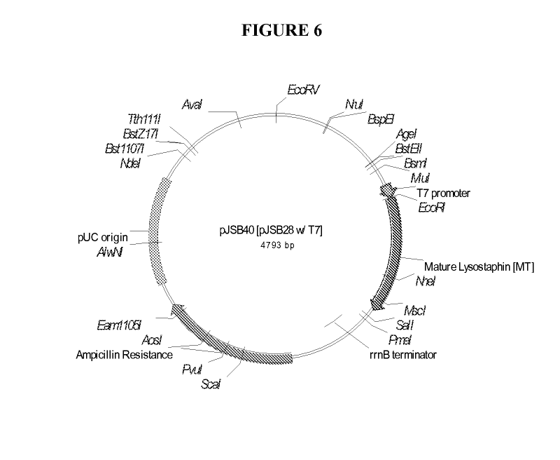

Genetic Modification of the Lysostaphin Gene. Plasmids were constructed for the expression of recombinant lysostaphin variants (e.g., displaying reduced immunogenicity) that displayed bactericidal activity. This was accomplished using synthesized, oligonucleotide pairs that, when annealed together, were designed to encode variant DNA sequences corresponding to amino acids 222-239 of lysostaphin. The paired oligonucleotides were further designed to comprise restriction endonuclease sites (e.g., MscI and SalI) for the cloning of this fragment into an expression vector, pJSB40 (See FIG. 6) for the expression of variant, full length lysostaphin.

pJSB40 was constructed by using the lysostaphin expression plasmid pJSB28 (See, U.S. Patent App. Pub. No. 20050118159) and replacing the arabinose-based expression upstream control elements with T7-based expression upstream control elements. Polymerase Chain Reaction (PCR) was used to amplify a fragment of the T7 promoter from the plasmid pET3A (Novagen) and the resulting fragment cloned into pJSB28. This generated a T7 controlled expression plasmid for intracellar expression of lysotaphin.

The PCR amplification reaction for the 5′ fragment contained 10 ng of template DNA (pET3A), 10 pmoles of primers JSBX-64 and JSBX-65 (GGTTCCGGATCCCGCGAAATTAATACG (SEQ ID NO. 99) and GTTTAACTTTAAGAAGGAGGAATTCACATGAAAAAAC (SEQ ID NO. 100), respectively)), 2.5 units of ExTaq polymerase (PanVera), 1× ExTaq reaction buffer, 200 μM dNTP, and 2 mM MgCl₂ in a 50 μl reaction volume. The template was denatured by an initial incubation at 96° C. for 3 min. The products were amplified by 25 thermal cycles 96° C. for 30 sec., 56° C. for 30 sec., 72° C. for 30 seconds. The PCR amplification reaction for the 3′ fragment contained 10 ng of template DNA (pJSB28), 10 pmoles of primers JSBX-66 and JSBX-67 (AGGAGGAATTCACATGAAAAAACTGCTGTTCGC (SEQ ID NO. 101) and AGTGAAGCTAGCTGACTCTG (SEQ ID NO. 102), respectively), 2.5 units of ExTaq polymerase, 1× ExTaq reaction buffer, 200 μM dNTP, and 2 mM MgCl₂ in a 50 μl reaction volume. The template was denatured by an initial incubation at 96° C. for 3 min. The products were amplified by 25 thermal cycles 96° C. for 30 sec., 52° C. for 30 sec., 72° C. for 30 seconds. PCR products were purified using the NUCLEOSPIN PCR Purification system (Clontech) per the manufacturer's procedure.

Overlap Extension (OLE)-PCR was then performed using equal amounts of 2 μL of the purified 5′ fragment and of the purified 3′ fragment (˜5 ul each), 10 pmol of primers JSBX-65 and JSBX-67, 2.5 units of ExTaq polymerase, 1× ExTaq reaction buffer, and 200 μM dNTP, 2 mM MgCl₂ in a 50 μl reaction volume. The template was denatured by an initial incubation at 96° C. for 3 min. The products were amplified by 30 thermal cycles 96° C. for 30 sec., 56° C. for 30 sec., 72° C. for 30 seconds. The first 5 cycles were performed without the addition of primer DNA to optimize the likelihood that the 5′ and 3′ sections, when denatured, would overlap appropriately to allow amplification of the entire lysostaphin sequence and promoter elements. The PCR products from successful reactions were purified using the NUCLEOSPIN PCR Purification system per manufacturer's instructions.

The PCR products were then digested with restriction endonucleases, BamHI and NheI, and cloned back into pJSB28 for protein expression. Ligations of the digested PCR fragments were carried out into de-phosphorylated, BamHI and NheI digested pJSB28, using ligase (Promega) and following the manufacturer's instructions using a 3:1 insert to vector molar ratio. One half (5 μl) of the ligation reactions was used to transform competent GC5 cells (GeneChoice) per the manufacturer's instructions. A cartoon depicting an expression plasmid, pJSB40, with unique restriction sites is provided in FIG. 6. MscI and SalI sites are located at the 3′ end of the lysostaphin encoding region.

pJSB40 was digested with restriction endonucleases MscI and SalI (New England Biolabs, Ipswich, Mass.) following the manufacturer's recommendations. The products of the digest were then size fractionated using agarose gel electrophoresis, and the large (4400 kb) DNA fragment was excised from the gel and purified. Synthetic oligonucleotide pairs were annealed and then ligated to the purified plasmid fragment using the FASTLINK DNA LIGATION SYSTEM (Epicentre, Madison, Wis.). Two microliters of each ligation was used to transform competent GC5 cells (PGC) as per the manufacturer's procedure. Bacterial clones containing plasmids with DNA inserts were identified using diagnostic PCR using primers that annealed 5′ and 3′ to the entire lysostaphin encoding region (JSBX-35 (CTATGCCATAGCATTTTTATCC (SEQ ID NO. 103) and JSBX-36 (CAAAACAGCCAAGCTGGAGACCG (SEQ ID NO. 104)).

Clones containing appropriately sized inserts (˜1000 bp) were chosen for DNA sequence evaluation. DNA sequencing was performed using cycle sequencing reactions primed by JSBX-60 (GCCAACGTATTTACTTGCCTGCAAAGACATGGAATAAATCTACTAATACTT) (SEQ ID NO. 105) and analyzed on a CEQ2000 capillary sequencer (Beckman/Coulter, Fullerton, Calif.). Once colonies were identified as comprising variant, full length lysostaphin coding sequence, the identified colonies were picked, cultured in growth media and prepared for plasmid DNA isolation. Plasmid DNA was confirmed via sequencing for each variant. The plasmid DNAs were then used to create E. coli expression strains via transformation of E. coli (BL21 (DE3) pLysS host cells) and for cell-free protein expression using the PROTEOMASTER RAPID TRANSLATION SYSTEM (Roche, Indianapolis, Ind.).

Cell-Free Production of Lysostaphin Variants. Reaction solutions of the RTS100 E. coli HY Kit (Roche, Indianapolis, Ind.) were prepared as per the manufacturer's instructions. The mixture contained all components necessary for transcription/translation in a cell-free system in the presence of DNA template. Briefly, 500 μg of the DNA template was used per 50 μl reaction. A plasmid encoding wild type lysostaphin served as a control. Bacterial cultures were grown at 30° C. for six hours with shaking. Expression of the lysostaphin variants was detected via Western blotting and compared to expression of wild type lysostaphin to verify expression of a full length lysostaphin variant.

S. carnosus Optical Density (O.D.) prop Activity Assays. An overnight culture of S. carnosus was washed with Phosphate Buffered Saline (PBS). A suspension of bacteria was then prepared, having an optical density at wave length 650 nm (OD₆₅₀) of 1.5-1.56, in PBS. The lysostaphin control and samples were diluted to approximately 50 μg/ml as determined by OD₂₈₀ (extinction coefficient for lysostaphin is 0.49 at OD₂₈₀). An initial “time zero” reading was taken on 576 μl of the cell suspension at OD₆₅₀, 24 μl of the sample or control was then added and mixed. The final concentration of lysostaphin in the samples was 2 μg/ml and the OD₆₅₀ of the samples were measured every 30 seconds for 30 min.

To compare the samples, a 50% OD drop time was used. Activity of the sample equaled: Time of 50% OD drop for the Standard, divided by Time of 50% OD drop for the Sample, multiplied by 100%.

Pilot Shake Flask Expression and PEI purification. Competent E. coli BL21 LysS (Novagen, San Diego, Calif.) cells were transformed with expression plasmids encoding the lysostaphin variants, according to the manufacturer's recommendation. Transformed cells were identified by antibiotic selection on agar plates containing ampicillin (100 μg/mL) and chloramphenicol (30 μg/mL). Isolated, single colonies were used to start overnight seed cultures in 1× “Fermentation Broth” ((Each four liters of media comprises 88 g glycerol, 72 g Yeastolate (Biospringer 1105C/180), 200 ml trace elements solution (1 ml concentrated sulfuric acid, 1.5 g Iron II sulfate heptahydrate, 3.5 g calcium chloride dihyrdrate, 0.62 g manganese sulfate monohydrate, 0.19 g zinc sulfate heptahydrate, 0.04 g copper sulfate 5-hydrate, and DI water), 0.5 ml Mazu DF204 antifoam (BASF), 6 g Citric acid, anhydrous, 13.6 g Potassium phosphate, monobasic, 6 g Magnesium sulfate heptahydrate (0.7H₂O), DI water to 4 L and adjust pH to 6.8 with NH₄OH and sterilize using 0.2 μM filter.) supplemented with ampicillin and chloramphenicol.

Expression cultures (15 ml of 1× “Fermentation Broth”) were inoculate with 0.3 ml of the seed culture. The cultures were grown at 37° C., 250 RPM until OD₆₀₀ reached between 0.5 and 1.0. Lysostaphin expression was then induced by the addition of isopropyl-β-D-thiogalactopyranosid (IPTG) to a final concentration of 1 mM. After addition of IPTG, the cultures were grown 4-5 hours.

Cells were harvested by centrifugation. Cell pellets were resuspended in 1 ml of buffer (10 mM Sodium Phosphate, 140 mM Sodium Chloride, pH 6.5). Cell suspensions were sonicated, on ice, for 1 min of total time with 10 sec ON/OFF intervals, using an ultrasonic cell disrupter VIRSONIC 600 VIRTIS (model 274506) with a ⅛″ tip. Sonicated lysates were clarified using PEI.

Lysates were diluted 1/40 (up to 0.5%) with 20% PEI, mixed and then incubated at RT for ˜30 min. Insoluble material was removed by centrifugation, spinning for 10 minutes at ˜14,000 rpm. Supernatants were transferred to clean tubes. Typical lysostaphin concentrations in these samples ranged from 0.5-1 mg/ml. Samples were diluted with “Lysostaphin Final Buffer” (pH 6.5, 10 mM Sodium phosphate, 140 mM NaCl) 20 fold (to about 50 μg/ml) and filtered through a 0.45 μm filter. Lysostaphin concentrations were determined using HPLC using 50 μg/ml of pure lysostaphin as a standard. Lysostaphin variants obtained were used for animal experiments.

Immunization of C57BL/6N mice with lysostaphin variants/evaluation of the immunogenicity of lysostaphin variants. 219N12 (hypo-immunized lysostaphin). The following procedure was used to evaluate the immunogenicity of lysostaphin variants. In order to obtain control serum, tail bleeds were collected from mice a day before the first injection (termed “normal mouse serum”). Lysostaphin variants were injected at two concentrations (2.5 μg or 50 mg combined with 5 μg of cholera toxin (CT) adjuvant). Mice were bled from tail on day 14 to obtain serum samples. ELISA was used to test serum for anti-lysostaphin antibodies. Mice were boosted with the same doses of the same lysostaphin variant with CT on day 15. Mice were bled from tail on day 29 to obtain serum samples. ELISA was used to test serums for anti-lysostaphin antibodies.

ELISA to detect lysostaphin-antibodies. One hundred μl of rabbit polyclonal anti-lysostaphin serum (Biocon, Inc, rabbit 1109) was diluted 1:10,000 in PBS and used to coat wells of a 96-well microtiter plate (NUNC, Rochester, N.Y.) overnight at 4° C. The plates were then washed with PBS and blocked with 100 μl/well of 1% BSA in PBS at room temperature for 30-60 minutes. Experimental samples and the lysostaphin standard (AMBICIN, Ambi) were diluted in PBS with 0.01% Tween and 0.1% BSA (PBS-T-BSA). The anti-lysostaphin coated, blocked plates were then washed with PBS-T four times. The samples and standard dilutions were then transferred (100 ul/well) onto anti-lysostaphin coated plate and incubated for 30-60 minutes. The plate was then washed 4 times with PBS-T. The detection antibody (polyclonal Rb anti-Lysostaphin, Rabbit 1109, Dec. 7, 2000; biotinylated, compound 3085, 1.6 mg/ml) was then diluted 1:800 in PBS-T-BSA and added at 100 uL/well. The plate was incubated 30-60 minutes at RT and then washed 4 times with PBS-T. ExtraAvidin-HRP (Sigma Cat# E2886) was diluted 1:8000 in PBS-T-BSA and then 100 uL/well was added to the plate which was incubated for 30-60 minutes. The plate was washed 4 times with PBS-T. One hundred μL/well of TMB-Microwell Substrate (BioFx Cat# TMBW 0100-01) was added and the reaction was allowed to proceed for 3-5 minutes before being stopped by the addition of TMB Stop reagent (BioFx Cat# STPR 0100-01). Absorbance was then read at 450 nm.

### Example 2

### Analysis of Lysostaphin Peptide Sequences

A complete analysis of overlapping 12-mer peptide sequences across the entire lysostaphin sequence was conducted. An algorithm (EPIMATRIX algorithm, EPIVAX, Inc., Providence, R.I.) was used to identify lysostaphin T-cell epitopes. These 12-mer peptides were analyzed against 8 common human MHC class II alleles for their ability to be bound by any of these class II alleles. Only those peptides sequences with resulting EPIMATRIX Z-Scores ≧1.64 were selected for further evaluation (See FIG. 4). Forty-nine such frames were identified as having at least one “hit,” many of which fell in close proximity to each other to form a “cluster.” Eight such clusters were identified that comprised 79% of the total number of predicted hits. Of these, four clusters (LYS030, LYS070, LYS108, and LYS219) contained the highest number of positive “hits”.

### Example 3

### Characterization of the Immunogenicity of Lysostaphin Peptide Sequences

In order to evaluate the immunogenicity of each of the 8 predicted clusters referenced in Example 2, an elispot assay using lysostaphin-exposed blood was performed. Briefly, a microtiter plate was coated with anti-lysostaphin antibody and then the various lysostaphin peptides were added. Human peripheral blood mononuclear cells (PBMC) were added to the wells, and interferon-γ production was quantified. The level of IFN-γ production indicated the level of T-cell activation elicited by the various peptides and correlated to their levels of immunogenicity. The results of these assays revealed that the regions with the highest predicted immunogenic potential (LYS030, LYS070, LYS108, and LYS219) contained significant T-cell epitopes. Aggressively modified variants of these peptides with amino acid substitutions in positions identified as likely to contribute to class II MHC binding lead to substantially reduced immunogenicity compared to their original wild type counterparts (See, e.g., Example 4, and FIGS. 4, 5, and 7). Additional variant peptides were then predicted utilizing more structurally conservative changes that would decrease the epitope content of lysostaphin with less chance of negatively affecting bacteriocidal activity. 3D modeling studies were performed to predict whether or not the proposed amino acid changes would be expected to have detrimental effects on the overall structure of the variants.

### Example 4

### De-Immunized Lysostaphin Variants are Less Immunogenic than Wild-Type Lysostaphin

Immunization of C57BL/6N mice with lysostaphin variants and evaluation of the immunogenicity of lysostaphin variants. 219N12 (de-immunized lysostaphin). The following procedure was used to evaluate the immunogenicity of lysostaphin variants. In order to obtain control serum, tail bleeds were collected from mice a day before the first injection (termed “normal mouse serum”). Lysostaphin variants were injected at two concentrations (2.5 μg or 50 μg combined with 5 μg of cholera toxin (CT) adjuvant). Mice were bled from tail on day 14 to obtain serum samples. ELISA was used to test serum for anti-lysostaphin antibodies. Mice were boosted with the same doses of the same lysostaphin variant with CT on day 15. Mice were bled from tail on day 29 to obtain serum samples. ELISA was used to test serums for anti-lysostaphin antibodies (as described in Example 1).

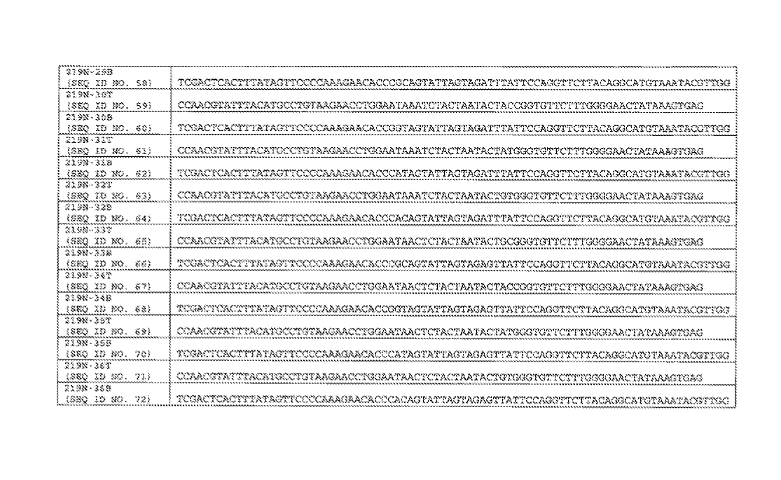

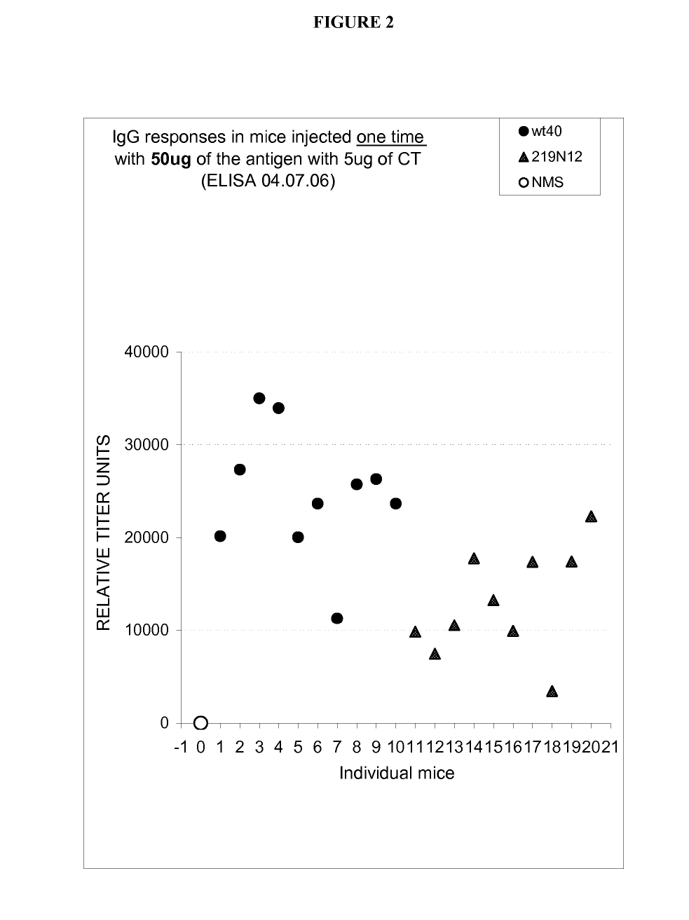

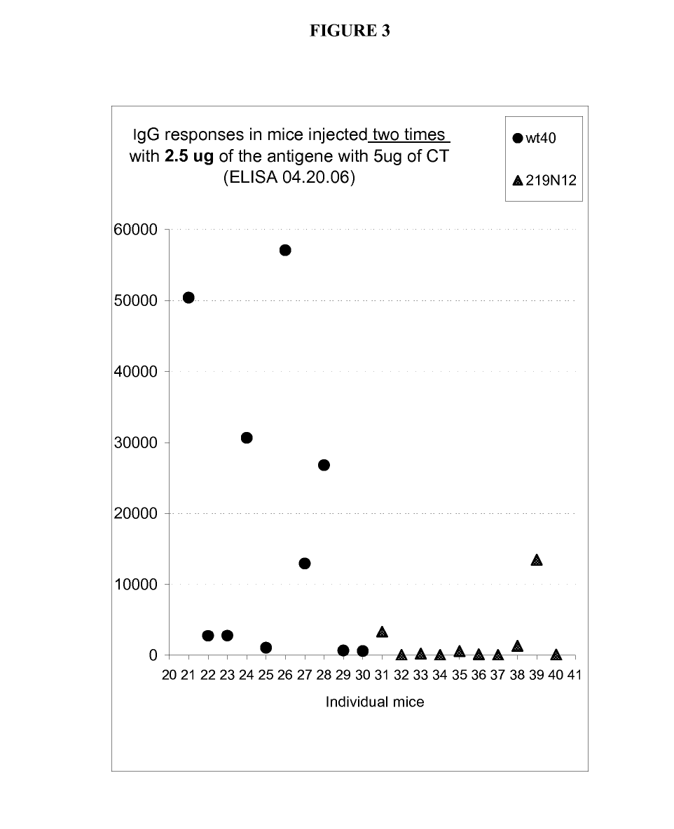

Mice administered de-immunized lysostaphin (e.g., variant 12 (219 (N12) comprising SEQ ID NO. 84) displayed significantly less IgG response than mice administered wild-type lysostaphin (See FIGS. 2 and 3). This was true whether administered one time with 50 μg lysostaphin plus 5 μg of CT, or whether injected twice with 2.5 μg of lysostaphin plus 5 μg CT. Thus, the present invention provides that de-immunized lysostaphin when administered to a subject elicits less of an immune response in the subject compared to the immune response elicited by non de-immunized lysostaphin (e.g., as determined by measuring IgG levels in the host).

### Example 5

### Identification and Characterization of Lysostaphin Variants

Experiments were conducted during development of the present invention to determine if the modification of immunogenic “hotspots” reduced the immunogenicity of the molecule while at the same time preserving bactericidal activity. Through the extensive analysis of mutational variants described in Examples 2 and 3, it was determined that a number of lysostaphin variants were generated that displayed both reduced immunogenicity (e.g., as measured by anti-lysostaphin antibody production), and bactericidal activity (See FIG. 5). For example, a number of variants displayed minimum bactericidal concentrations (i.e., the concentration of variant required to cause a >3 log 10 drop in S. aureus strain ATCC 49521 within 10 minutes (See, e.g., Kusuma and Kokai-Kun 2005. Antimicrobial Agents and Chemotherapy 49:3256-3263)), displayed minimum inhibitory concentrations (i.e., the concentration of variant required to prevent visible growth of S. aureus strain ATCC 49521 for 24 hrs (See, e.g., See, e.g., Kusuma and Kokai-Kun 2005. Antimicrobial Agents and Chemotherapy 49:3256-3263)), displayed in vivo activity (i.e., dose of variant required to clear S. aureus infection by strain ATCC 49521 when administered once a day for 3 days in a mouse infection model), and/or displayed similar kinetics for biofilm reduction (i.e., the time, in hours, to reduce the optical density at 650 nM of an established S. aureus biofilm to 50% of the starting OD using 12.5 μg/ml of variant (See, e.g., Wu et al 2003 Antimicrob. Agents Chemother. 47:3407-3414)) comparable to that of wild type lysostaphin (See FIG. 5).

In vivo activity, 6 week-old female CF-1 mice were challenged i.v. with ˜2×10⁷ CFU of either MSSA (ATCC 49521) or MRSA (NRS123). Three hours post challenge, treatments commenced. Mice were treated once a day for 3 days with an i.v. dose of truncated lysostaphin (See U.S. Patent App. Pub. No. 20050118159) or purified variant. The mice were sacrificed on day 6 and infection of the liver, spleen and kidneys were determined by mechanically disrupting the organs and plating them on solid media. Results of the variants were compared with the truncated lysostaphin results at the same dose.

Thus, the present invention provides lysostaphin variants with reduced immunogenicity (See Examples 2-3) that also retain bacteriocidal activity.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising de-immunized lysostaphin.

2. The composition of claim 1, wherein said de-immunized lysostaphin comprises SEQ ID NO. 74.

3. The composition of claim 1, wherein said de-immunized lysostaphin comprises SEQ ID NO. 108.

4. The composition of claim 1, wherein said de-immunized lysostaphin comprises one or more variant lysostaphin sequences, wherein the one or more variant sequences are selected from the group consisting of SEQ ID NOs. 73, 75-98, 106, 107 and 109.

5. The composition of claim 1, wherein said de-immunized lysostaphin is capable of cleaving cross-linked polyglycine bridges in the cell wall peptidoglycan of staphylococci.

6. The composition of claim 1, wherein said de-immunized lysostaphin is recombinantly produced.

7. The composition of claim 1, wherein said de-immunized lysostaphin possesses a terminal cysteine.

8. The composition of claim 1, wherein said de-immunized lysostaphin is conjugated to a water-soluble polymer.

9. The composition of claim 8, wherein said water-soluble polymer is selected from the group consisting of poly(alkylene oxides), polyoxyethylated polyols and poly(vinyl alcohols).

10. The composition of claim 9, wherein said poly(alkylene oxide) is polyethylene glycocl (PEG).

11. A pharmaceutical composition for treating microbial infection comprising de-immunized lysostaphin and a pharmaceutically acceptable carrier.

12. The pharmaceutical composition of claim 11, wherein said de-immunized lysostpahin is less immunogenic than non-de-immunized lysostaphin.

13. The pharmaceutical composition of claim 11, wherein said de-immunized lysostaphin is capable of cleaving the cross-linked polyglycine bridges in the cell wall peptidoglycan of staphylococci.

14. The pharmaceutical composition of claim 11, further comprising an antibiotic.

15. The pharmaceutical composition of claim 14, wherein said antibiotic is selected from the group consisting of β-lactams, cephalosporins, aminoglycosides, sulfonamides, antifolates, macrolides, quinolones, glycopeptides, polypeptides and combinations thereof.

16. A method for the prophylactic or therapeutic treatment of a microbial infection in a subject comprising administering to said subject a pharmaceutical composition comprising de-immunized lysostaphin and a pharmaceutically acceptable carrier, in an amount effective for preventing or treating said infection.

17. The method of claim 16, wherein said infection is a bacterial infection.

18. The method of claim 16, wherein said bacterial infection is caused by bacteria from the genus Staphylococcus.

19. The method of claim 18, wherein said bacteria comprises Staphylococcus aureus.

20. The method of claim 18, wherein said bacteria comprises Staphylococcus epidermidis.


SEQUENCE LISTING (117 sequences)

SEQ ID NO: 1 - DNA, 80 bp, Staphylococcus simulans
ccaacgtggctacttgcctgtaagaacatggaataactctactaatactgcgggtaccctttggggaactataaagtgag

SEQ ID NO: 2 - DNA, 84 bp, Staphylococcus simulans
tcgactcactttatagttccccaaagggtacccgcagtattagtagagttattccatgttcttacaggcaagtagccacgttgg

SEQ ID NO: 3 - DNA, 80 bp, Staphylococcus simulans
ccaacgtggctacgcgcctgtaagaacatggaataactctactaatactgcgggtaccctttggggaactataaagtgag

SEQ ID NO: 4 - DNA, 84 bp, Staphylococcus simulans
tcgactcactttatagttccccaaagggtacccgcagtattagtagagttattccatgttcttacaggcgcgtagccacgttgg

SEQ ID NO: 5 - DNA, 80 bp, Staphylococcus simulans
ccaacgtggctacgcgcctgcaagaacatggaataactctactaatactgcgggtaccctttggggaactataaagtgag

SEQ ID NO: 6 - DNA, 84 bp, Staphylococcus simulans
tcgactcactttatagttccccaaagggtacccgcagtattagtagagttattccatgttcttgcaggcgcgtagccacgttgg

SEQ ID NO: 7 - DNA, 80 bp, Staphylococcus simulans
ccaacgtggctacttgcctgtaagaacatggaataactctactaatactctgggtaccctttggggaactataaagtgag

SEQ ID NO: 8 - DNA, 84 bp, Staphylococcus simulans
tcgactcactttatagttccccaaagggtacccagagtattagtagagttattccatgttcttacaggcaagtagccacgttgg

SEQ ID NO: 9 - DNA, 80 bp, Staphylococcus simulans
ccaacgtggctacgcgcctgtaagaacatggaataactctactaatactctgggtaccctttggggaactataaagtgag

SEQ ID NO: 10 - DNA, 84 bp, Staphylococcus simulans
tcgactcactttatagttccccaaagggtacccagagtattagtagagttattccatgttcttacaggcgcgtagccacgttgg

SEQ ID NO: 11 - DNA, 80 bp, Staphylococcus simulans
ccaacgtggctacgcgcctgcaagaacatggaataactctactaatactctgggtaccctttggggaactataaagtgag

SEQ ID NO: 12 - DNA, 84 bp, Staphylococcus simulans
tcgactcactttatagttccccaaagggtacccagagtattagtagagttattccatgttcttgcaggcgcgtagccacgttgg

SEQ ID NO: 13 - DNA, 80 bp, Staphylococcus simulans
ccaacgtatttacttgcctgtaagacattggaataaatctactaatactgcgggtgttctttggggaactataaagtgag

SEQ ID NO: 14 - DNA, 84 bp, Staphylococcus simulans
tcgactcactttatagttccccaaagaacacccgcagtattagtagatttattccaatgtcttacaggcaagtaaatacgttgg

SEQ ID NO: 15 - DNA, 80 bp, Staphylococcus simulans
ccaacgtatttacttgcctgtaagacattggaataactctactaatactgcgggtgttctttggggaactataaagtgag

SEQ ID NO: 16 - DNA, 84 bp, Staphylococcus simulans
tcgactcactttatagttccccaaagaacacccgcagtattagtagagttattccaatgtcttacaggcaagtaaatacgttgg

SEQ ID NO: 17 - DNA, 80 bp, Staphylococcus simulans
ccaacgtatttacttgcctgtaagacattggaataaatctactaatactatgggtgttctttggggaactataaagtgag

SEQ ID NO: 18 - DNA, 84 bp, Staphylococcus simulans
tcgactcactttatagttccccaaagaacacccatagtattagtagatttattccaatgtcttacaggcaagtaaatacgttgg

SEQ ID NO: 19 - DNA, 80 bp, Staphylococcus simulans
ccaacgtatttacttgcctgtaagacattggaataactctactaatactatgggtgttctttggggaactataaagtgag

SEQ ID NO: 20 - DNA, 84 bp, Staphylococcus simulans
tcgactcactttatagttccccaaagaacacccatagtattagtagagttattccaatgtcttacaggcaagtaaatacgttgg

SEQ ID NO: 21 - DNA, 80 bp, Staphylococcus simulans
ccaacgtatttacttgcctgtaagacattggaataaatctactaatactaccggtgttctttggggaactataaagtgag

SEQ ID NO: 22 - DNA, 84 bp, Staphylococcus simulans
tcgactcactttatagttccccaaagaacaccggtagtattagtagatttattccaatgtcttacaggcaagtaaatacgttgg

SEQ ID NO: 23 - DNA, 80 bp, Staphylococcus simulans
ccaacgtatttacttgcctgtaagacattggaataactctactaatactaccggtgttctttggggaactataaagtgag

SEQ ID NO: 24 - DNA, 84 bp, Staphylococcus simulans
tcgactcactttatagttccccaaagaacaccggtagtattagtagagttattccaatgtcttacaggcaagtaaatacgttgg

SEQ ID NO: 25 - DNA, 80 bp, Staphylococcus simulans
ccaacgtatttacttgcctgtaagacattggaataaatctactaatactgtgggtgttctttggggaactataaagtgag

SEQ ID NO: 26 - DNA, 84 bp, Staphylococcus simulans
tcgactcactttatagttccccaaagaacacccacagtattagtagatttattccaatgtcttacaggcaagtaaatacgttgg

SEQ ID NO: 27 - DNA, 80 bp, Staphylococcus simulans
ccaacgtatttacttgcctgtaagacattggaataactctactaatactgtgggtgttctttggggaactataaagtgag

SEQ ID NO: 28 - DNA, 84 bp, Staphylococcus simulans
tcgactcactttatagttccccaaagaacacccacagtattagtagagttattccaatgtcttacaggcaagtaaatacgttgg

SEQ ID NO: 29 - DNA, 80 bp, Staphylococcus simulans
ccaacgtatttacattcctgtaagaacctggaataaatctactaatactgcgggtgttctttggggaactataaagtgag

SEQ ID NO: 30 - DNA, 84 bp, Staphylococcus simulans
tcgactcactttatagttccccaaagaacacccgcagtattagtagatttattccaggttcttacaggaatgtaaatacgttgg

SEQ ID NO: 31 - DNA, 80 bp, Staphylococcus simulans
ccaacgtggctacttgcctgtaagaacatggaataactctactaatactctgggtgcgctttggggaactataaagtgag

SEQ ID NO: 32 - DNA, 84 bp, Staphylococcus simulans
tcgactcactttatagttccccaaagcgcacccagagtattagtagagttattccatgttcttacaggcaagtagccacgttgg

SEQ ID NO: 33 - DNA, 80 bp, Staphylococcus simulans
ccaacgtggctacgcgcctgtaagaacatggaataactctactaatactctgggtgcgctttggggaactataaagtgag

SEQ ID NO: 34 - DNA, 84 bp, Staphylococcus simulans
tcgactcactttatagttccccaaagcgcacccagagtattagtagagttattccatgttcttacaggcgcgtagccacgttgg

SEQ ID NO: 35 - DNA, 80 bp, Staphylococcus simulans
ccaacgtggctacgcgcctgcaagaacatggaataactctactaatactctgggtgcgctttggggaactataaagtgag

SEQ ID NO: 36 - DNA, 84 bp, Staphylococcus simulans
tcgactcactttatagttccccaaagcgcacccagagtattagtagagttattccatgttcttgcaggcgcgtagccacgttgg

SEQ ID NO: 37 - DNA, 80 bp, Staphylococcus simulans
ccaacgtggctacttgcctgtaagaacatggaataactctactaatactgcaggtgcgctttggggaactataaagtgag

SEQ ID NO: 38 - DNA, 84 bp, Staphylococcus simulans
tcgactcactttatagttccccaaagcgcacctgcagtattagtagagttattccatgttcttacaggcaagtagccacgttgg

SEQ ID NO: 39 - DNA, 80 bp, Staphylococcus simulans
ccaacgtggctacgcgcctgtaagaacatggaataactctactaatactgcaggtgcgctttggggaactataaagtgag

SEQ ID NO: 40 - DNA, 84 bp, Staphylococcus simulans
tcgactcactttatagttccccaaagcgcacctgcagtattagtagagttattccatgttcttacaggcgcgtagccacgttgg

SEQ ID NO: 41 - DNA, 80 bp, Staphylococcus simulans
ccaacgtggctacgcgcctgcaagaacatggaataactctactaatactgcaggtgcgctttggggaactataaagtgag

SEQ ID NO: 42 - DNA, 84 bp, Staphylococcus simulans
tcgactcactttatagttccccaaagcgcacctgcagtattagtagagttattccatgttcttgcaggcgcgtagccacgttgg

SEQ ID NO: 43 - DNA, 80 bp, Staphylococcus simulans
ccaacgtatttacattcctgtaagaacctggaataaatctactaatactaccggtgttctttggggaactataaagtgag

SEQ ID NO: 44 - DNA, 84 bp, Staphylococcus simulans
tcgactcactttatagttccccaaagaacaccggtagtattagtagatttattccaggttcttacaggaatgtaaatacgttgg

SEQ ID NO: 45 - DNA, 80 bp, Staphylococcus simulans
ccaacgtatttacattcctgtaagaacctggaataaatctactaatactatgggtgttctttggggaactataaagtgag

SEQ ID NO: 46 - DNA, 84 bp, Staphylococcus simulans
tcgactcactttatagttccccaaagaacacccatagtattagtagatttattccaggttcttacaggaatgtaaatacgttgg

SEQ ID NO: 47 - DNA, 80 bp, Staphylococcus simulans
ccaacgtatttacattcctgtaagaacctggaataaatctactaatactgtgggtgttctttggggaactataaagtgag

SEQ ID NO: 48 - DNA, 84 bp, Staphylococcus simulans
tcgactcactttatagttccccaaagaacacccacagtattagtagatttattccaggttcttacaggaatgtaaatacgttgg

SEQ ID NO: 49 - DNA, 80 bp, Staphylococcus simulans
ccaacgtatttacattcctgtaagaacctggaataactctactaatactgcgggtgttctttggggaactataaagtgag

SEQ ID NO: 50 - DNA, 84 bp, Staphylococcus simulans
tcgactcactttatagttccccaaagaacacccgcagtattagtagagttattccaggttcttacaggaatgtaaatacgttgg

SEQ ID NO: 51 - DNA, 80 bp, Staphylococcus simulans
ccaacgtatttacattcctgtaagaacctggaataactctactaatactaccggtgttctttggggaactataaagtgag

SEQ ID NO: 52 - DNA, 84 bp, Staphylococcus simulans
tcgactcactttatagttccccaaagaacaccggtagtattagtagagttattccaggttcttacaggaatgtaaatacgttgg

SEQ ID NO: 53 - DNA, 80 bp, Staphylococcus simulans
ccaacgtatttacattcctgtaagaacctggaataactctactaatactatgggtgttctttggggaactataaagtgag

SEQ ID NO: 54 - DNA, 84 bp, Staphylococcus simulans; identical to SEQ ID NO: 52

SEQ ID NO: 55 - DNA, 80 bp, Staphylococcus simulans
ccaacgtatttacattcctgtaagaacctggaataactctactaatactgtgggtgttctttggggaactataaagtgag

SEQ ID NO: 56 - DNA, 84 bp, Staphylococcus simulans
tcgactcactttatagttccccaaagaacacccacagtattagtagagttattccaggttcttacaggaatgtaaatacgttgg

SEQ ID NO: 57 - DNA, 80 bp, Staphylococcus simulans
ccaacgtatttacatgcctgtaagaacctggaataaatctactaatactgcgggtgttctttggggaactataaagtgag

SEQ ID NO: 58 - DNA, 84 bp, Staphylococcus simulans
tcgactcactttatagttccccaaagaacacccgcagtattagtagatttattccaggttcttacaggcatgtaaatacgttgg

SEQ ID NO: 59 - DNA, 80 bp, Staphylococcus simulans
ccaacgtatttacatgcctgtaagaacctggaataaatctactaatactaccggtgttctttggggaactataaagtgag

SEQ ID NO: 60 - DNA, 84 bp, Staphylococcus simulans
tcgactcactttatagttccccaaagaacaccggtagtattagtagatttattccaggttcttacaggcatgtaaatacgttgg

SEQ ID NO: 61 - DNA, 80 bp, Staphylococcus simulans
ccaacgtatttacatgcctgtaagaacctggaataaatctactaatactatgggtgttctttggggaactataaagtgag

SEQ ID NO: 62 - DNA, 84 bp, Staphylococcus simulans
tcgactcactttatagttccccaaagaacacccatagtattagtagatttattccaggttcttacaggcatgtaaatacgttgg

SEQ ID NO: 63 - DNA, 80 bp, Staphylococcus simulans
ccaacgtatttacatgcctgtaagaacctggaataaatctactaatactgtgggtgttctttggggaactataaagtgag

SEQ ID NO: 64 - DNA, 84 bp, Staphylococcus simulans
tcgactcactttatagttccccaaagaacacccacagtattagtagatttattccaggttcttacaggcatgtaaatacgttgg

SEQ ID NO: 65 - DNA, 80 bp, Staphylococcus simulans
ccaacgtatttacatgcctgtaagaacctggaataactctactaatactgcgggtgttctttggggaactataaagtgag

SEQ ID NO: 66 - DNA, 84 bp, Staphylococcus simulans
tcgactcactttatagttccccaaagaacacccgcagtattagtagagttattccaggttcttacaggcatgtaaatacgttgg

SEQ ID NO: 67 - DNA, 80 bp, Staphylococcus simulans
ccaacgtatttacatgcctgtaagaacctggaataactctactaatactaccggtgttctttggggaactataaagtgag

SEQ ID NO: 68 - DNA, 84 bp, Staphylococcus simulans
tcgactcactttatagttccccaaagaacaccggtagtattagtagagttattccaggttcttacaggcatgtaaatacgttgg

SEQ ID NO: 69 - DNA, 80 bp, Staphylococcus simulans
ccaacgtatttacatgcctgtaagaacctggaataactctactaatactatgggtgttctttggggaactataaagtgag

SEQ ID NO: 70 - DNA, 84 bp, Staphylococcus simulans
tcgactcactttatagttccccaaagaacacccatagtattagtagagttattccaggttcttacaggcatgtaaatacgttgg

SEQ ID NO: 71 - DNA, 80 bp, Staphylococcus simulans
ccaacgtatttacatgcctgtaagaacctggaataactctactaatactgtgggtgttctttggggaactataaagtgag

SEQ ID NO: 72 - DNA, 84 bp, Staphylococcus simulans
tcgactcactttatagttccccaaagaacacccacagtattagtagagttattccaggttcttacaggcatgtaaatacgttgg

SEQ ID NO: 73 - protein, 23 aa, Staphylococcus simulans
SGQRIYIPVRTWNKSTNTAGVLW

SEQ ID NO: 74 - protein, 23 aa, Staphylococcus simulans
SGQRIYLPVRHWNKSTNTAGVLW

SEQ ID NO: 75 - protein, 23 aa, Staphylococcus simulans
SGQRIYLPVRHWNKSTNTTGVLW

SEQ ID NO: 76 - protein, 23 aa, Staphylococcus simulans
SGQRIYIPVRTWNKSTNTMGVLW

SEQ ID NO: 77 - protein, 23 aa, Staphylococcus simulans
SGQRIYLPVRHWNKSTNTMGVLW

SEQ ID NO: 78 - protein, 23 aa, Staphylococcus simulans
SGQRIYIPVRTWNKSTNTTGVLW

SEQ ID NO: 79 - protein, 23 aa, Staphylococcus simulans
SGQRIYIPVRTWNNSTNTTGVLW

SEQ ID NO: 80 - protein, 23 aa, Staphylococcus simulans
SGQRIYMPVRTWNKSTNTTGVLW

SEQ ID NO: 81 - protein, 23 aa, Staphylococcus simulans
SGQRIYLPVRHWNKSTNTVGVLW

SEQ ID NO: 82 - protein, 23 aa, Staphylococcus simulans
SGQRIYIPVRTWNKSTNTVGVLW

SEQ ID NO: 83 - protein, 23 aa, Staphylococcus simulans
SGQRIYLPVRHWNNSTNTAGVLW

SEQ ID NO: 84 - protein, 23 aa, Staphylococcus simulans
SGQRIYLPVRHWNNSTNTTGVLW

SEQ ID NO: 85 - protein, 23 aa, Staphylococcus simulans
SGQRIYLPVRHWNNSTNTMGVLW

SEQ ID NO: 86 - protein, 23 aa, Staphylococcus simulans
SGQRIYLPVRHWNNSTNTVGVLW

SEQ ID NO: 87 - protein, 23 aa, Staphylococcus simulans
SGQRIYIPVRTWNNSTNTMGVLW

SEQ ID NO: 88 - protein, 23 aa, Staphylococcus simulans
SGQRIYIPVRTWNNSTNTAGVLW

SEQ ID NO: 89 - protein, 23 aa, Staphylococcus simulans
SGQRIYIPVRTWNNSTNTVGVLW

SEQ ID NO: 90 - protein, 23 aa, Staphylococcus simulans
SGQRIYMPVRTWNKSTNTAGVLW

SEQ ID NO: 91 - protein, 23 aa, Staphylococcus simulans
SGQRIYMPVRTWNKSTNTMGVLW

SEQ ID NO: 92 - protein, 23 aa, Staphylococcus simulans
SGQRIYMPVRTWNKSTNTVGVLW

SEQ ID NO: 93 - protein, 23 aa, Staphylococcus simulans
SGQRIYMPVRTWNNSTNTTGVLW

SEQ ID NO: 94 - protein, 23 aa, Staphylococcus simulans
SGQRIYMPVRTWNNSTNTMGVLW

SEQ ID NO: 95 - protein, 23 aa, Staphylococcus simulans
SGQRIYMPVRTWNNSTNTAGVLW

SEQ ID NO: 96 - protein, 23 aa, Staphylococcus simulans
SGQRIYMPVRTWNNSTNTVGVLW

SEQ ID NO: 97 - protein, 23 aa, Staphylococcus simulans
SGQRIYLPVRTWNKSTNTLGVLW

SEQ ID NO: 98 - protein, 23 aa, Staphylococcus simulans
SGQRGYLPVRTWNDSTNTLGVLW

SEQ ID NO: 99 - DNA, 27 bp, Staphylococcus simulans
ggttccggatcccgcgaaattaatacg

SEQ ID NO: 100 - DNA, 37 bp, Staphylococcus simulans
gtttaactttaagaaggaggaattcacatgaaaaaac

SEQ ID NO: 101 - DNA, 33 bp, Staphylococcus simulans
aggaggaattcacatgaaaaaactgctgttcgc

SEQ ID NO: 102 - DNA, 20 bp, Staphylococcus simulans
agtgaagctagctgactctg

SEQ ID NO: 103 - DNA, 22 bp, Staphylococcus simulans
ctatgccatagcatttttatcc

SEQ ID NO: 104 - DNA, 23 bp, Staphylococcus simulans
caaaacagccaagctggagaccg

SEQ ID NO: 105 - DNA, 51 bp, Staphylococcus simulans
gccaacgtatttacttgcctgcaaagacatggaataaatctactaatactt

SEQ ID NO: 106 - protein, 25 aa, Staphylococcus simulans
QRGYLPVRTWNNSTNTLGTLWGTIK

SEQ ID NO: 107 - protein, 25 aa, Staphylococcus simulans
QRGYAPVRTWNNSTNTLGTLWGTIK

SEQ ID NO: 108 - protein, 25 aa, Staphylococcus simulans
QRIYLPVRHWNKSTNTVGVLWGTIK

SEQ ID NO: 109 - protein, 25 aa, Staphylococcus simulans
QRIYLPVRHWNNSTNTVGVLWGTIK

SEQ ID NO: 110 - DNA, 89 bp, Staphylococcus simulans
tggccaacgtggctacttgcctgtaagaacatggaataactctactaatactctgggtaccctttggggaactataaagtgagtcgacc

SEQ ID NO: 111 - DNA, 89 bp, Staphylococcus simulans
accggttgcaccgatgaacggacattcttgtaccttattgagatgattatgagacccatgggaaaccccttgatatttcactcagctgg

SEQ ID NO: 112 - DNA, 88 bp, Staphylococcus simulans
tggccaacgtggctacgcgcctgtaagaacatggaataactctactaatactctgggtaccctttggggaactataaagtgagtcgac

SEQ ID NO: 113 - DNA, 88 bp, Staphylococcus simulans
accggttgcaccgatgcgcggacattcttgtaccttattgagatgattatgagacccatgggaaaccccttgatatttcactcagctg

SEQ ID NO: 114 - DNA, 89 bp, Staphylococcus simulans
tggccaacgtatttacttgcctgtaagacattggaataaatctactaatactgtgggtgttctttggggaactataaagtgagtcgacc

SEQ ID NO: 115 - DNA, 89 bp, Staphylococcus simulans
accggttgcataaatgaacggacattctgtaaccttatttagatgattatgacacccacaagaaaccccttgatatttcactcagctgg

SEQ ID NO: 116 - DNA, 88 bp, Staphylococcus simulans
tggccaacgtatttacttgcctgtaagacattggaataactctactaatactgtgggtgttctttggggaactataaagtgagtcgac

SEQ ID NO: 117 - DNA, 88 bp, Staphylococcus simulans
accggttgcataaatgaacggacattctgtaaccttattgagatgattatgacacccacaagaaaccccttgatatttcactcagctg